(19) United States
(12) Patent Application Publication
(10) Pub. No.: US 2009/0117179 A1
(43) Pub. Date: May 7, 2009

(54) siDNA against Influenza Virus

(76) Inventor: Karin Molling, Berlin (DE)

Correspondence Address: JOYCE VON NATZMER;PEQUIGNOT + MYERS LLC, 200 Madison Avenue, Suite 1901, New York, NY, 10016, US

(21) Appl. No.: 12/203,179

(22) Filed: Sep. 3, 2008

(30) Foreign Application Priority Data
Sep. 3, 2007 (EP) 07075751.3

(51) Int. Cl. A61K 9/127; C07H 21/04; A61K 31/711; A61K 31/7105; A61K 35/76

(52) U.S. Cl. 424/450; 536/24.5; 514/44; 424/93.6

(57) ABSTRACT

Silencing of Influenza virus RNA can be achieved by siDNA. These are oligodeoxynucleotides having an antisense-strand homologous to the viral RNA and a second strand, partially complementary to the antisense-strand. The two strands are preferentially linked by a linker (eg 4 thymidines). Triple-helix formation with the target RNA is a preferred effect. The siDNA is superior to siRNA because it acts earlier, is easier taken up by the cell, the formation of RNA-DNA hybrids is preferred over double-stranded DNA or double-stranded RNA, which forms as tertiary structures in RNA genomes. Also the induction of interferon is less likely. siDNA is easier to synthesize and it is more stable. It can be combined with siRNA.

This application claims priority under 35 USC 119(b) to European Patent Application No. 07075751.3, filed Sep. 3, 2007, which is incorporated herein by reference in its entirety.

### FIELD OF THE INVENTION

Silencing of Influenza Virus RNA can be achieved by siDNA. These are oligodeoxynucleotides comprising an antisense-strand homologous to the viral RNA and a second strand, partially complementary to the antisense-strand. The two strands are preferentially linked by a linker (e.g., 4 thymidines). Triple-helix formation between siDNA and the target RNA is a preferred effect. The siDNA is superior to siRNA because the formation of RNA-DNA hybrids is preferred over double-stranded DNA or double-stranded RNA, which forms as tertiary structure in RNA genomes. Also the induction of interferon is less likely. Furthermore, siDNA is effective at earlier time-points after addition to the cell than siRNA. Therefore viral RNA is targeted by siDNA before it is amplified, while siRNA targets the about 1000 fold higher viral RNA population of the progeny. siDNA is easier to synthesize and is more stable. It can be combined with other siDNAs targeted against various strains in a cocktail; it can also be combined with siRNA to target early and late steps in viral replication. siDNA can be targeted to any virus strain, including the present avian pandemic strain.

The publications and other materials referenced throughout the present application, including, among others, patents, are incorporated herein by reference in their entireties.

### BACKGROUND OF THE INVENTION

Influenza virus is a member of the Orthomyxoviruses causing wide-spread infection in the human respiratory tract, but existing vaccines and drug therapy are of limited value. In a typical year 20% of the human population is afflicted by the virus, resulting in 40,000 deaths. In one of the most devastating human catastrophies in history, at least 20 million people died worldwide during the 1918 Influenza A virus pandemic. The threat of a new influenza pandemic persists because existing vaccines or therapies are of limited value. In elderly the efficacy of vaccination is only about 40%. The existing vaccines have to be redesigned every year, because of genetic variation of the viral antigens, the Haemagglutinin HA and the Neuraminidase N. Four antiviral drugs have been approved in the United States for treatment and/or prophylaxis of Influenza. However, their use is limited because of severe side effects and the possible emergence of resistant viruses.

The siDNA, the basis for his invention, is a partially double-stranded hairpin-loop structured DNA oligonucleotide of about 20 to 50 nucleotides in length, whereby the antisense strand is fully homologous to the target viral RNA and the second strand only partially homologous to the antisense strand, forming a hairpin-loop structure. It forms an RNA-DNA hybrid with the antisense strand targeting the viral RNA. The siDNA may form triple-helix with the target RNA. An RNA-DNA hybrid is thermodynamically favored over double-stranded DNA; therefore the siDNA interacting with the viral RNA is preferred over RNA-RNA interactions.

The siDNA is highly sequence-specific, length-dependent and induces cleavage (silencing) of a target RNA by an RNase H (Moelling et al, FEBS Letters, 580, 3545-3550 (2006)), which is a component in some viruses but also in the cell. RNases H removes RNA primers required for the initiation of DNA synthesis or DNA replication in the nucleus or mitochondria, but they are also present in the cytoplasm. siDNA is related to siRNA, whereby the cleavage enzyme is highly related to the RNase H.

Recently RNA interference has been used, whereby a short double-stranded RNA, 21 to 25 nucleotides in length, siRNA, directs sequence-specific degradation of messenger mRNA. Synthetic siRNA confers transient interference of gene-expression in a sequence-specific manner. This has significant medical applications. Inhibition of Influenza virus replication by siRNA against the viral RNA encoding the nucleocapsid or one component of the polymerase, the RNA transcriptase (PA), holds promise as prophylaxis or therapy against influenza virus infection in humans (Ge et al, PNAS 100, 2718-1723 (2003)).

It was recently demonstrated by the inventor the use of siDNA targeted to the HIV genome. This activates the RNase H of the virus and leads to silencing of the viral RNA (Matzen et al, Nature Biotechnol. 25, 669-674 (2007)). The RNase H is a Hybrid-specific RNase which cleaves RNA in an RNA-DNA hybrid and was discovered by Moelling et al (Nature New Biology 234, 240-243 (1971)). The inventor also observed that cellular RNase H-like activities such as RNase H1 and RNase H2A, B, C and even Agonaute 2 contribute to siDNA-mediated silencing. Agonaute 2 (Ago2) is the enzyme known to induce siRNA-mediated silencing. The inventor showed that Ago2 is enzymatically related to RNases H and can induce siDNA-mediated silencing as well (Moelling et al. Cold Spring Harb. Symp. Quant. Biol. 71, 365-368 (2006), Small Regulatory RNAs).

Furthermore, it could be shown by the inventor that siDNA is able to induce silencing of an oncogenic retrovirus, the Spleen Focus-Forming Virus and prevent infection, cause delay of disease progression and lead to increased survival time (Nature Biotechnol. 25, 669-674 (2007)). Furthermore the inventor showed in several cases that siDNA is superior to a single-stranded antisense effect (Jendis et al. AIDS Research and Human Retroviruses 12, 1161-1168 (1996), AIDS Research and Human Retro-viruses 14, 999-1005, (1998), Moelling et al, FEBS Letters, 580, 3545-3550 (2006), Matzen et al. Nature Biotechnol. 25, 669-674 (2007)).

Thus the effect of siDNA has been demonstrated in several cases.

One problem to be solved with the present invention is to present an effective antiviral therapeutic against Influenza virus infections by siDNA.

This problem may be solved by the features of the independent claims.

An object of the invention is therefore to present siDNA oligonucleotides, capable of binding to one or more RNA target regions of Influenza virus, as antiviral therapeutic, whereby the siDNA oligonucleotides are comprising or consisting of an antisense-strand homologous to the viral RNA target and a second strand, partially complementary to the antisense-strand.

Another object of the invention is therefore to present siDNA oligonucleotides, capable of binding to one or more RNA target regions of Influenza virus, as antiviral therapeutic, whereby the siDNA oligonucleotides are comprising or consisting of an antisense-strand fully or almost fully homologous to the viral RNA target and a second strand, partially complementary to the antisense-strand forming a partially double stranded hairpin-loop-structured oligonucleotide, comprising G-clusters consisting of or including at least two G nucleotides in succession to allow tetrade or tetramer formation or tetra-helices or higher-ordered structures within the same siDNA oligonucleotide molecule (cis conformation) or through interaction with another siDNA oligonucleotide molecule (trans conformation).

Another object of the invention is to provide a method, respectively the use of siDNA oligonucleotides, or a combination of two or three siDNAs, as antiviral therapeutic for the treatment of Influenza virus infections, capable of binding to RNA target regions of Influenza or different Influenza virus variants, whereby different siDNA oligonucleotides may be combined in a cocktail as pharmaceutical agent.

A further object of the invention is to provide a pharmaceutical composition as antiviral therapeutic for the treatment of Influenza virus infections, comprising said siDNA oligonucleotides capable of binding to RNA target regions of Influenza virus as antiviral therapeutic.

### BRIEF DESCRIPTION OF THE FIGURES

Abbreviations used are as follows:

ODN IPA 5′: Oligodeoxynucleotide Influenza A Polymerase A, 5′ End

ODN IPA -PPT: Polypurine-rich

ODN IPAsi: target side used for siRNA

PB-AUG: Polymerase B subunit. start site (AUG)

Linkers are indicated as 4 Ts, or can also be symbolized by a curved line;

Linkers can also be modified by phosphothioate modifications

Stars (*) indicate phosphothioate-modifications

Vertical bars indicate Watson-Crick bonds

FIG. 1: Preferred sequences according to the invention (A to G)

FIG. 2: LOCUS EF467820 2233 bp cRNA linear VRL 11-MAR-2007 DEFINITION Influenza A virus (A/Puerto Rico/8/34(H1N1)) segment 3, polymerase PA,

complete sequence

underlined and bold are the targeted regions according to FIG. 1

FIG. 3: Polymerase A (PA) of Influenza H5N1 (from Pubmed (nucleotides) (A/Hong kong/156/97 (H5N1)) Accession number AJ 289874.1

underlined and bold are the targeted regions according to FIG. 1

FIG. 4: Preferred sequences according to the invention

FIG. 5: Influenza Virus was used to infect cells in tissue culture in the presence of ODNs against Influenza polymerase gene PA, three different regions (4, 5, 6, see FIG. 4). SiRNA was used as control and ODNA (FEBS.L. 580, 3545 (2006)) was used as an unspecific control. Lipofectamin was used to facilitate uptake. At the times indicated after infection the virus was determined in the supernatant by highly sensitive RT-PCR. Uninfected (mock-infected) cells served as another control.

FIG. 6: Sequences according to the experimental design of FIG. 7

FIG. 7: Determining the role of cellular RNase H1 and RNase H2A in the gene silencing effects of ODN A, asA and siA (see FIG. 3). FIG. 7 shows the experimantal design.

FIG. 8: Knockdown of cellular RNase H1 or RNase H2A in HEK293 significantly reduced the gene silencing effects of asA but not those of ODN A or siA in live cells

HEK293 cells were transfected with siNeg, siRNase H1 or siRNase H2A siRNA duplexes (2 nM) with HiPerfect for 48 h, after which the cells were trypsinised and re-seeded into 12-well plates at a density of 5×105 cells per well. After overnight incubation at 37° C., the cells were co-transfected with pEGFP-5′PPT (360 ng or 113 fmole) and the various oligonucleotides (100 nM) using Lipofectamine 2000 for 20 h before analysis by flow cytometry. The mean fluorescence intensities were normalized against those of control samples which were not treated with oligonucleotides.

### SUMMARY OF THE INVENTION

Therefore, siDNA oligonucleotides, capable of binding to one or more RNA target regions of Influenza virus are claimed as antiviral therapeutic, whereby the siDNA oligonucleotides comprise or are consisting of an antisense-strand homologous (or having a sequence identity as specified below) to the viral RNA target and a second strand, partially complementary (or partially identical) to the antisense-strand forming a hairpin-loop structure. These siDNA oligonucleotides may bind to one or more RNA target regions of Influenza virus. The siDNA oligonucleotides may correspond to one or more Influenza virus target regions of at least 20 nucleotides in length, wherein the antisense strand of siDNA is fully or almost fully (more than 80%, more preferably more than 90%) homologous or fully or almost fully identical to the target Influenza viral RNA. Conserved target RNA regions are preferred. Further, the second strand of the siDNA oligonucleotides is preferably connected to the antisense strand through a thymidine linker, preferred—but not exclusively—4 nucleotides in length, and wherein the second strand is partially (40-60% homology) complementary (or partially identical) to the antisense-strand and may be able to form triple helices by non-Watson-Crick base pairing with the viral RNA target strand.

One object of the invention is the use of siDNA as antiviral therapeutic. These are oligodeoxynucleotides comprising or consisting of an antisense-strand homologous (more than 80%, more preferably more than 90%) to or having a certain sequence identity (see below, but preferably more than 80%, and even more preferably more than 90%) with the viral RNA and a second strand, partially (40-60%) complementary (or partially identical) to the antisense-strand. The siDNA may be targeted to a conserved region of Influenza RNA, 20 to 25 nucleotides or longer, which is fully or almost fully homologous (or fully or almost fully identical) to the target RNA. A second strand may be connected to the antisense strand through a Thymidine linker, e.g. 4 nucleotides in length. The second strand may be partially complementary to the antisense-strand and may be able to form triple helices by non-Watson-Crick base pairing.

Also a preferred embodiment of the invention is the use of a combination of two or three siDNAs as described herein as antiviral therapeutic capable of binding to RNA target regions of Influenza virus as pharmaceutical agent. Further preferred is the use of a cocktail of different siDNA oligonucleotides to target different Influenza virus variants.

Influenza virus in the meaning of this invention is not restricted to Influenza A virus, Influenza B virus or Influenza C virus, but also includes any virulent human pathogens of Influenza types. Therefore, other Influenza viruses such as avian Influenza virus are also an object of the invention. The serotypes that have been confirmed in humans, ordered by the number of known human pandemic deaths, are:
H1N1 caused “Spanish Flu.”
H2N2 caused “Asian Flu.”
H3N2 caused “Hong Kong Flu.”
H5N1 is a pandemic threat in 2006-7 flu season.
H7N7 has unusual zoonotic potential.
H1N2 is endemic in humans and pigs.
H9N2, H7N2, H7N3, H10N7.

The siDNA is preferably applied to an infected cell or an infected individual with a transducing agent, wherein as transducing agent may be selected from the following group: the virus itself, a replicating Influenza virus particle, which carries the siDNA into the cell during the process of infection, a liposome, transmembrane carriers, peptides. Most preferred is a cell transfection by using a lipid-based transfection reagent like Lullaby which is known from siRNA silencing. However, as the person skilled in the art will appreciate, other lipid based transducing agents are also within the scope of the present invention.

The siDNA oligonucleotides according to the invention are preferred as antiviral therapeutic as part of a pharmaceutical composition comprising these siDNA oligonucleotides capable of binding to RNA target regions of Influenza virus or its variants.

The preferred target site on the viral RNA is highly conserved, but generally at least conserved to a certain degree. Targeting a subunit of the transcriptase and its silencing would reduce viral RNA replication efficiently.

It is an unexpected unique embodiment of the invention that it acts early on the virus replication cycle and holds promise as a prevention of infection. It could be applied as nasal spray and prevent virus propagation in the body, the effect called viremia. Alternatively it may be used to essentially prevent (i.e., the virus infection proceeds without any clinical signs of virus infection). This cannot be accomplished by siRNA.

siDNA can also be targeted to cellular mRNAs coding for proteins essential for virus replication and indirectly prevent Influenza virus replication.

### DETAILED DESCRIPTION OF VARIOUS AND PREFERRED EMBODIMENTS OF THE INVENTION

Under the term “homology” the following should be understood: A siDNA oligonucleotide according to the invention comprises an antisense-strand which is more than 80% homologous to the viral RNA target and a second strand, which is partially complementary to the antisense-strand, forming a partially double stranded hairpin-loop-structure. The term “partially complementary” means a homology between 40 and 60% within the hairpin-loop.

The term “sequence identity” refers to a measure of the identity of the nucleotide sequences. In general, the sequences are aligned so that the highest order match is obtained. “Identity”, per se, has recognized meaning in the art and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Whether any particular nucleic acid sequence, say an antisense-strand, is more than 50%, 60%, 70%, 75%, 80%, 85%, 90%, 95%, 96%, 97%, 98% or 99% identical to, for instance, the viral target can be determined conventionally using known computer programs such as DNAsis software (Hitachi Software, San Bruno, Calif.) for initial sequence alignment followed by ESEE version 3.0 DNA/protein sequence software (cabot@trog.mbb.sfu.ca) for multiple sequence alignments. The term “partially identical” means a sequence identity between 40 and 60% within the hairpin-loop. The term “almost fully identical” means more than 80%, more preferably more than 90% identical.

Under the term “G-cluster” the following should be understood:

“G-cluster” according to the invention are motifs within a sequence of a siDNA oligonucleotide. A G-cluster is defined by at least 2 G nucleotides in succession (“GG”). Several G-clusters lead to tetrade or tetramer formation or tetra-helices or higher-ordered structures within the siDNA oligonucleotide molecule. Each G-cluster is separated from another G-Cluster by 1 to 20× nucleotides, wherein X is A, T, C or a single G, followed by A, T or C. General examples (among others) according to said definition are

| | |
|---|---|
| i. | GG |
| ii. | GGG |
| iii. | GGXG |
| iv. | GGGG |
| v. | GGXXXGGXXXXGG |
| vi. | GGXXGGXXXXGGG |

G-tetrade formation may be beneficial consisting e.g. of GGXXGGXXXXGGG etc, whereby X represents basically other nucleotides than “GG”, such as A, T or C or a single G, as long as the single G is followed by A, T or C. The siDNA oligonucleotides according to the invention contain G clusters to allow tetrade or tetramer formation or tetra-helices by those sequences. The hairpin-loop structure may comprise or consist of preferably of up to 9 non-self-complementary sequences at the 3′- and 5′-ends, followed by 6base-pairing, 2 non-pairing, 3 pairing, 2 non-pairing, 3 pairing sequences, followed by 4Ts (see for proof of principle Moelling et al, FEBS Letters, 580, 3545-3550 (2006)). A homology or sequence identity of 40 to 60% is preferred.

The tetrade or tetramer formation or tetra-helices or higher-ordered structures within the siDNA oligonucleotide molecule results of an interaction of G-clusters within the same siDNA oligonucleotide molecule (so-called cis conformation) or through interaction with another siDNA oligonucleotide molecule (so-called trans conformation). Both conformations (cis or trans) are possible.

Specific examples according to the invention are (framed sequence motifs are able to tetrade or tetramer formation or tetra-helices or higher-ordered structures; bold nucleotides indicate G-cluster):

ODN IPA (= SEQ ID NO 12):
3′-TTTGTTACTTTCTCATACCCCTCCTTTTTA [symbol] GGTTGAGAATCG
TTTTTT-5′
ODN IPA-PPT (= SEQ ID NO 13)
3′-TTTTACTTTACCCCTTACCTCTACTTTTTAGTTCAG [symbol]
GGTTTTCGTTTT-5′
ODN IPAsi (= SEQ ID NO 14)
3′-ATACTTCGTTAACTCCTCACGGACTTTTTAGTTTGTGTCGAGTTATTC
TTCGTG-5′
ODN PB-AUG (= SEQ ID NO 15)
3′-GTAAACTTACCTACAGTTA [symbol] TTTTCCATTCTATTTGTA [symbol]
TT-5′

The invention is based on the cognition that a DNA strand has a thermodynamic preference to form an RNA-DNA hybrid over double-stranded DNA or double-stranded RNA. Thus the invention is based on the not expected result that siDNA is of high preference (in contrast to siRNA) to form RNA-DNA hybrids; siDNA is therefore of advantage and a preferred object of the invention. Furthermore siDNA acts against the newly infecting incoming viral RNA, 2 to 3 days before siRNA. Thus a virus infection can be prevented by siDNA only. In contrast thereto, interferon-induction is a risk for siRNA and reduced or absent for an RNA-DNA hybrid or double-stranded DNA. Furthermore, siDNA is preferred due to the fact that DNA is easier to synthesize and more stable. Also, it can be combined with siRNA.

The siDNA may also be a chimeric molecule, consisting of ribonucleotides and desoxyribonucleotides. However, the RNase H cleavage site needs to consist of a local RNA-DNA hybrid.

siDNA is also superior to a simple antisense oligodeoxynucleotide, because it is more stable during uptake and inside the cell and therefore more effective. It acts earlier than antisense DNA, and can target incoming RNA, while antisense DNA targets mRNA (Jekerle, V. et. al. J. Pharm. Sci. 8, 516-527 (2005)).

It was shown before that siDNA is targeting viral RNA before replication while siRNA is effective only late during replication targeting the mRNA (Westerhout, E. M. ter Brake, O. and Berkhout, B. Retrovirology 3,57-65 (2006)).

The invention takes advantage of this effect in silencing of Influenza Virus RNA. Using siDNA oligonucleotides is significantly more effective early during infection in contrast to siRNA. An unexpected advantage in conjunction with the present invention of siDNA versus siRNA is the targeting of incoming viral RNA before viral replication while siRNA is effective only late during replication targeting the mRNA. At this stage the RNA is highly amplified (e.g. 1000 fold or more) while the incoming RNA consists of a single copy. Viral RNA is destroyed within about 8 to 14 hours post infection by a 5- to 10 fold reduction. At this time point the siRNA effect is almost undetectable. The siRNA needs to enter a multi-protein complex, RISC, to prepare the cleavage, a process which takes about 3 days. By then the virus has replicated and spread through the organism efficiently. After 30 to 40 hours the reduction of viral RNA is similar in both cases, amounting to about 3- to 4 fold reduction.

The RNA silencing inside the cell is achieved by the cellular RNases H, such as RNase H1 or RNase H2a, b, c. Also the Ago2, the siRNA-inducing silencing enzyme, is RNase H-like. These enzymes are located mainly in the nucleus but also in the cytoplasm. They are mainly responsible for removing RNA primers during DNA replication in the cell. The siDNA will lead to silencing of the viral RNA during early stages of replication and/or mRNA during late stages of replication, which requires higher doses. Treatment is—not limited to—performed by topical application, cream, spray, drops or injection of siDNA into the blood stream or intraperitoneally. Depending on the stability of the compound the treatment can be repeated. Toxicity has not been detected in mouse studies (Matzen et al. Nature Biotech. 25, 669-674(2007)).

As stated above, it could be shown by the inventor that siDNA is able to induce silencing of an oncogenic retrovirus, the Spleen Focus-Forming Virus and prevent infection, cause delay of disease progression and lead to increased survival time (Nature Biotechnology 25, 669-674 (2007)). The mechanism was due to the action of the viral RNase H. A contribution of cellular RNases H or related cellular enzymes is likely. New unpublished results show that a reporter plasmid can be targeted by a siDNA and that the gene expression is silenced by RNase H1 or RNase H2a. This was demonstrated by inactivation of these RNases H, which strongly reduces the silencing effects.

Preferred embodiments of the invention are described as follows:

Similar to siRNA a combination of two or three siDNAs may be targeted to different regions of the RNA genome or a cocktail may be designed to target different Influenza Virus variants. These include avian strains as e.g. the present avian pandemic strain H5N1. The preferred siDNA sequences are connected by a linker, preferably a thymidine linker, in particular a T4-linker.

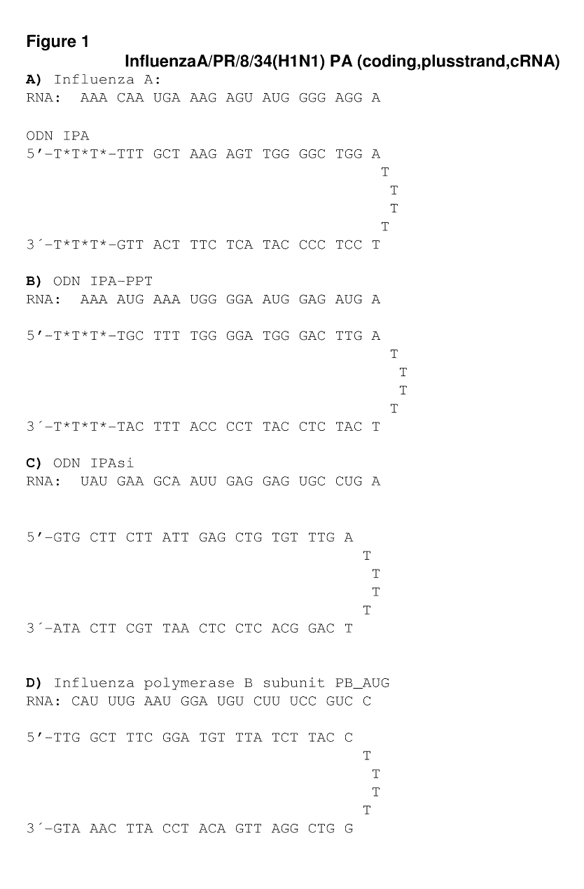

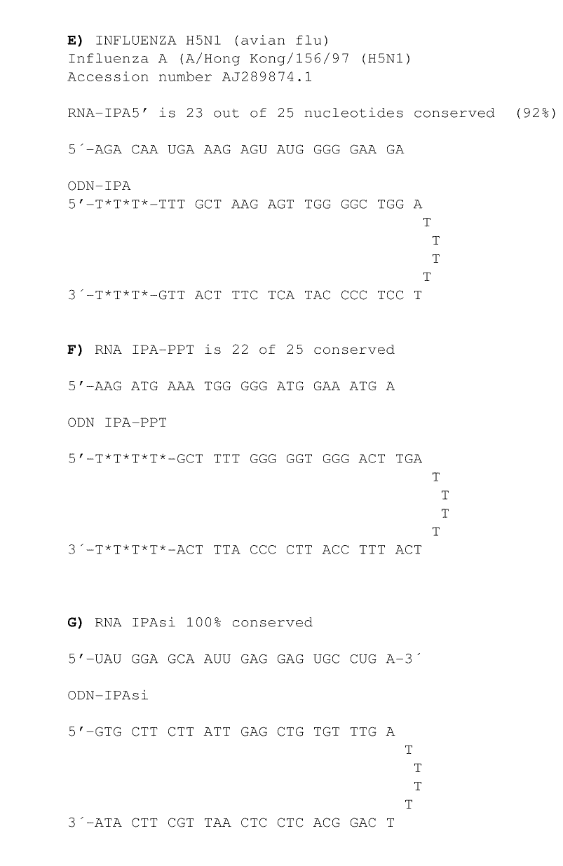

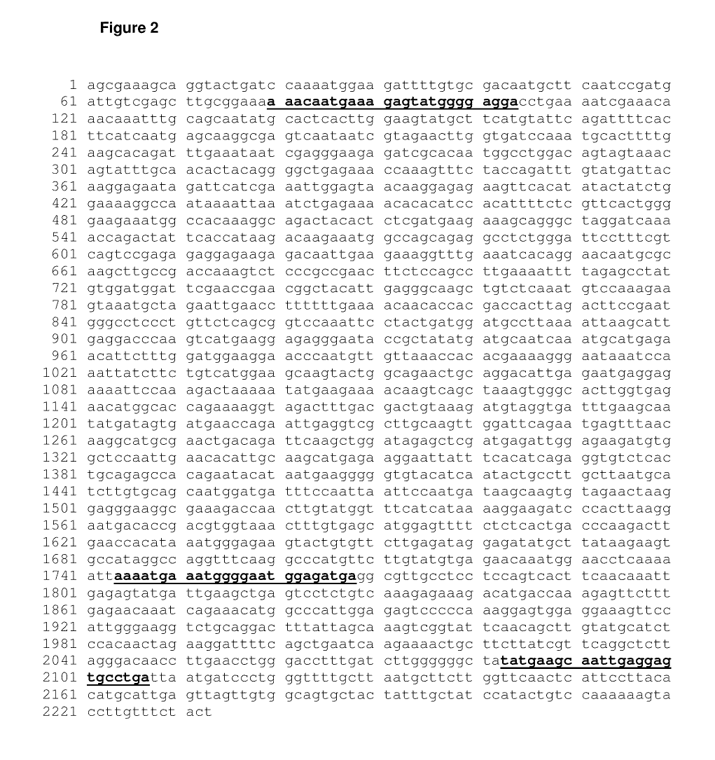

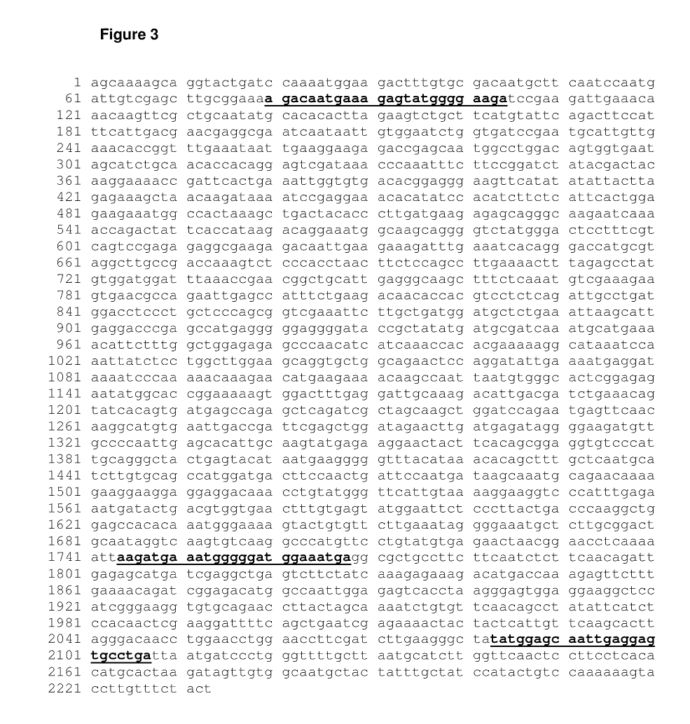

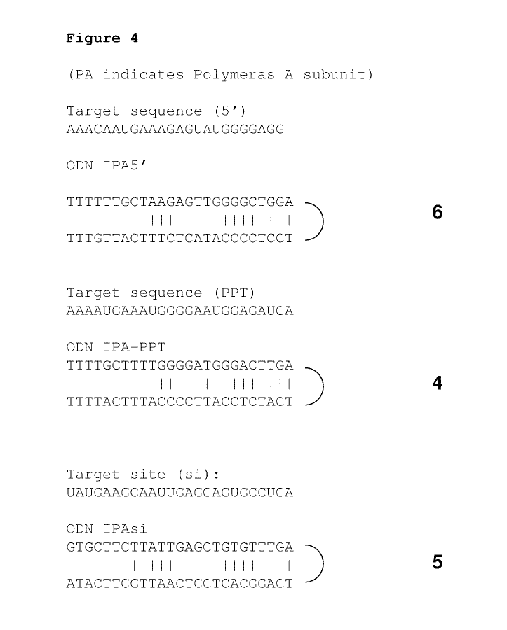

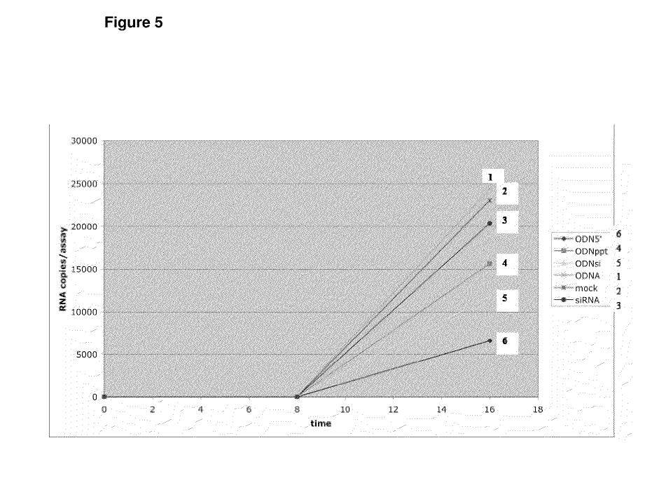

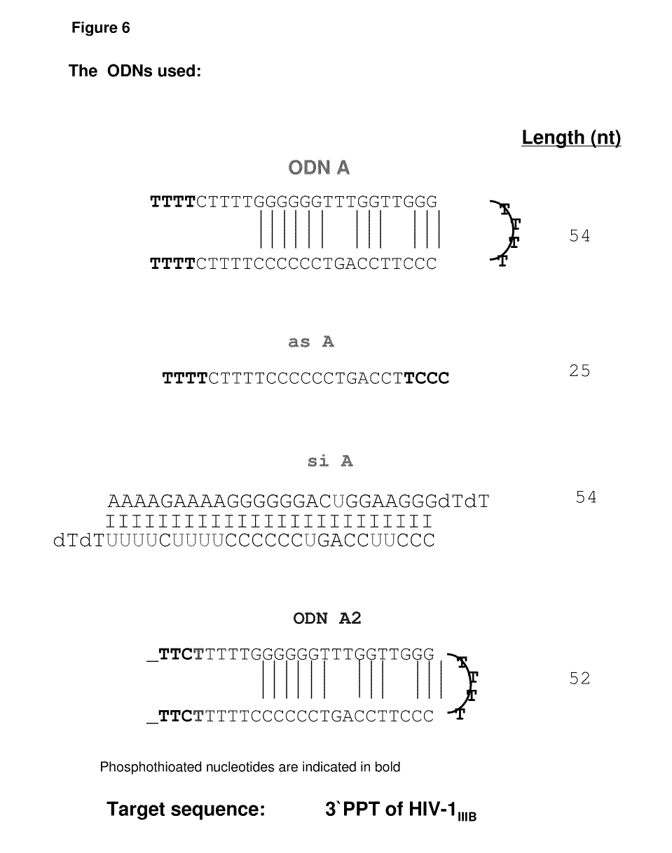

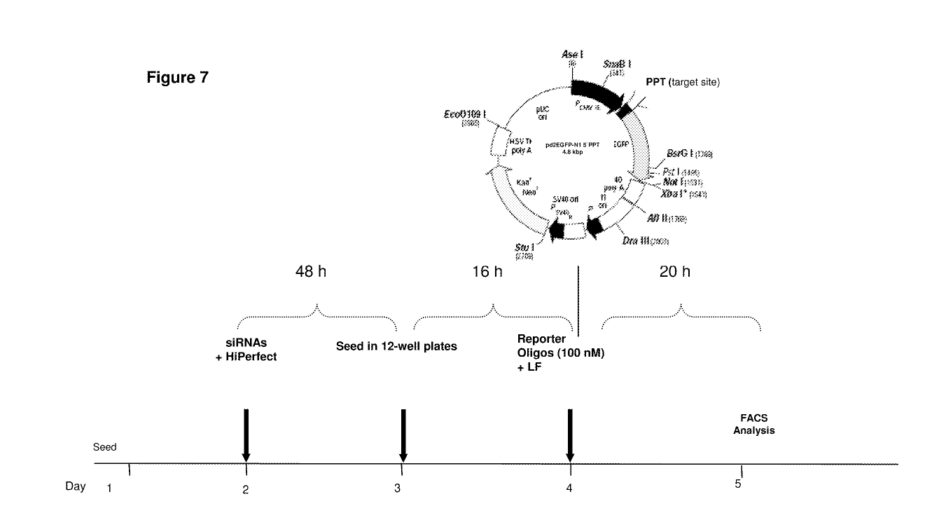

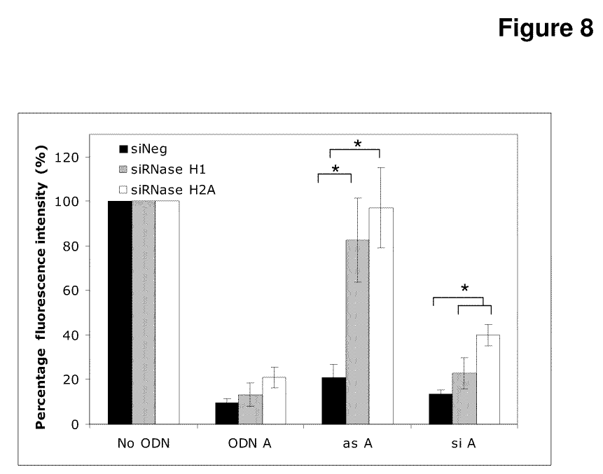

Preferred according to the invention are the sequences for siDNA as shown in FIG. 1, which are also an object of the invention and listed below:

| | Sequence | |
|---|---|---|
| Sequence | name | Sequence |
| SEQ ID | Target | 5′-AAACAAUGAAAGAGUAUGGGGAGG-3′ |
| NO 1 | Sequence | |
| | (RNA) | |
| SEQ ID | ODN IPA5′ | 5′-TTTTTTGCTAAGAGTTGGGGCTGGA-3′ |
| NO 2 | (siDNA)- | |
| | antisense | |
| SEQ ID | ODN IPA5′ | 3′-TTTGTTACTTTCTCATACCCCTCCT-5′ |
| NO 3 | (siDNA)- | |
| | second | |
| | strand | |
| SEQ ID | Target | 5′-AAAAUGAAAUGGGGAAUGGAGAUGA-3′ |
| NO 4 | Sequence | |
| | PPT (RNA) | |
| SEQ ID | ODN IPA-PPT | 5′-TTTTGCTTTTGGGGATGGGACTTGA-3′ |
| NO 5 | (siDNA)- | |
| | antisense | |
| SEQ ID | ODN IPA-PPT | 3′-TTTTACTTTACCCCTTACCTCTACT-5′ |
| NO 6 | (siDNA)- | |
| | second | |
| | strand | |
| SEQ ID | Target | 5′-UAUGAA |
| NO 7 | Sequence si | [symbol] |
| | (RNA) | |
| SEQ ID | ODN IPAsi | 5′-GTGCTTCTTATTGAGCTGTGTTTGA-3′ |
| NO 8 | (siDNA)- | |
| | antisense | |
| SEQ ID | ODN IPAsi | 3′-ATACTTCGTTAACTCCTCACGGACT-5′ |
| NO 9 | (siDNA)- | |
| | second | |
| | strand | |
| SEQ ID | siDNA | |
| NO 12 | Oligo- | |
| | nucleotide | |
| | ODN IPA | |
| SEQ ID | siDNA | |
| NO 13 | Oligo- | |
| | nucleotide | |
| | ODN IPA-PPT | |
| SEQ ID | siDNA | |
| NO 14 | Oligo- | |
| | nucleotide | |
| | ODN IPAsi | |
| SEQ ID | siDNA | |
| NO 15 | Oligo- | |
| | nucleotide | |
| | ODN PB-AUG | |

The siDNA “antisense” strands and siDNA second strands are linked via a thymidine (T4) linker.

The siDNA may be stabilized by base modifications e.g. thioates at the ends and/or in the center. The invention is not restricted to those kinds of base modifications. Other base modifications, i.e phosphorothioates, P-DNA, sugar-phopsphate modifications, Morpholinos, amidates, 2′-OMe, 2′-F, 2′-MOE, LNA and further are also preferred without limitation to those modifications. According to the invention in particular siDNA oligonucleotides comprising at least one sequence selected from the Group: SEQ ID NO 12, SEQ ID NO 13, SEQ ID NO 14 or SEQ ID NO 15 are preferred for the use as antiviral therapeutic for the treatment of Hepatitis C virus (HCV) infections and for a corresponding pharmaceutical composition.

I claim:

1. Isolated and purified siDNA oligonucleotides for binding to one or more RNA target regions of Influenza virus comprising
an antisense-strand homologous to the viral RNA target and
a second strand, partially complementary to the antisense-strand, wherein said siDNA oligonucleotides are for binding one or more RNA target regions of Influenza virus.

2. Isolated and purified siDNA oligonucleotides for binding to one or more RNA target regions of Influenza virus comprising
an antisense-strand fully or almost fully homologous to the viral RNA target and
a second strand, partially complementary to the antisense-strand, forming a partially double stranded hairpin-loop-structured oligonucleotide comprising G-clusters, wherein the G-clusters include at least two G nucleotides in succession to allow tetrade or tetramer formation or tetra-helices or higher-ordered structures within a certain siDNA oligonucleotide molecule (cis conformation) or through interaction with another siDNA oligonucleotide molecule (trans conformation).

3. Isolated and purified siDNA oligonucleotides according to claim 1 or 2, wherein one or more siDNA oligonucleotides correspond to one or more conserved Influenza target regions, of at least 20 nucleotides in length, wherein the antisense strand of siDNA is more than 80%, more preferably more than 90% homologous to the target Influenza viral RNA

4. Isolated and purified siDNA oligonucleotides according claim 1 or 2, wherein the second strand is connected to the antisense strand through a thymidine linker, preferably 4 nucleotides in length, and wherein the second strand is, with a homology of 40 to 60%, partially complementary within the hairpin-loop to the antisense-strand and adapted to form triple helices by non-Watson-Crick base pairing with the viral RNA target strand.

5. Isolated and purified siDNA oligonucleotides according to claim 1, wherein said siDNA comprise G-clusters including at least two G nucleotides in succession to allow tetrade or tetramer formation or tetra-helices or higher-ordered structures within a certain siDNA oligonucleotide molecule (cis conformation) or through interaction with another siDNA oligonucleotide molecule (trans conformation).

6. Isolated and purified siDNA oligonucleotides according to claim 2 or 5, wherein at least two of the G-clusters are separated from each other by other nucleotides like A, T, C or G.

7. Isolated and purified siDNA oligonucleotides according to the preceding claims, wherein the siDNA is stabilized by base modifications.

8. Isolated and purified siDNA oligonucleotides according to 1 or 2, selected from sequences SEQ ID NO 12, SEQ ID NO 13, SEQ ID NO 14 or SEQ ID NO 15.

9. Method of treating Influenza virus infection in an individual or cell in need thereof comprising:
administering siDNA, or a combination of two or three siDNAs according to the preceding claims in an Influenza virus infection treating effective amount to an individual or cell in need thereof, wherein said siDNA, or said combination of two or three siDNAs binds to RNA target regions of Influenza or different Influenza virus variants.

10. The method of claim 9, wherein the siDNA also contains ribonucleotides, siDNA/RNA chimeras, or is combined with siRNAs.

11. The method of claims 9 or 10, wherein a cocktail of different siDNA oligonucleotides is administered and the different siDNA oligonucleotides target different Influenza virus variants.

12. The method of claim 9 or 10, wherein the siDNA is applied to an infected cell or an infected individual with a transducing agent.

13. The method of claim 12, wherein the transducing agent is selected from the following group: the virus itself, a replicating Influenza virus particle which carries the siDNA into the cell during the process of infection, a liposome, transmembrane carriers or peptides.

14. Pharmaceutical composition comprising
siDNA oligonucleotides according to claim 1 or 2 for binding to RNA target regions of Influenza virus as antiviral therapeutic, wherein said pharmaceutical composition is an antiviral therapeutic for the treatment of Influenza virus infections.

15. Pharmaceutical composition according to claim 14, comprising at least one sequence of the group: SEQ ID NO 12, SEQ ID NO 13, SEQ ID NO 14 or SEQ ID NO 15.


SEQUENCE LISTING (15 sequences)

SEQ ID NO: 1 - RNA, 25 nt, artificial
  target sequence (RNA)
aaacaaugaaagaguauggggagga

SEQ ID NO: 2 - DNA, 25 bp, artificial
  ODN IPA5′ (siDNA) - antisense
ttttttgctaagagttggggctgga

SEQ ID NO: 3 - DNA, 25 bp, artificial
  ODN IPA5′ (siDNA) - second strand
tcctccccatactctttcattgttt

SEQ ID NO: 4 - RNA, 25 nt, artificial
  Target Sequence PPT (RNA)
aaaaugaaauggggaauggagauga

SEQ ID NO: 5 - DNA, 25 bp, artificial
  ODN IPA-PPT (siDNA) - antisense
ttttgcttttggggatgggacttga

SEQ ID NO: 6 - DNA, 25 bp, artificial
  ODN IPA-PPT (siDNA) - second strand
tcatctccattccccatttcatttt

SEQ ID NO: 7 - RNA, 25 nt, artificial
  Target Sequence si (RNA)
uaugaagcaauugaggagugccuga

SEQ ID NO: 8 - DNA, 25 bp, artificial
  ODN IPAsi (siDNA)- antisense
gtgcttcttattgagctgtgtttga

SEQ ID NO: 9 - DNA, 25 bp, artificial
  ODN IPAsi (siDNA)- second strand
tcaggcactcctcaattgcttcata

SEQ ID NO: 10 - DNA, 2233 bp, Influenza A virus segment 3, polymerase PA
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagattttcacttcatcaatgagcaaggcgagtcaataatcgtagaacttggtgatccaaatgcacttttgaagcacagatttgaaataatcgagggaagagatcgcacaatggcctggacagtagtaaacagtatttgcaacactacaggggctgagaaaccaaagtttctaccagatttgtatgattacaaggagaatagattcatcgaaattggagtaacaaggagagaagttcacatatactatctggaaaaggccaataaaattaaatctgagaaaacacacatccacattttctcgttcactggggaagaaatggccacaaaggcagactacactctcgatgaagaaagcagggctaggatcaaaaccagactattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctactgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaagaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagtttaacaaggcatgcgaactgacagattcaagctggatagagctcgatgagattggagaagatgtggctccaattgaacacattgcaagcatgagaaggaattatttcacatcagaggtgtctcactgcagagccacagaatacataatgaagggggtgtacatcaatactgccttgcttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacataaatgggagaagtactgtgttcttgagataggagatatgcttataagaagtgccataggccaggtttcaaggcccatgttcttgtatgtgagaacaaatggaacctcaaaaattaaaatgaaatggggaatggagatgaggcgttgcctcctccagtcacttcaacaaattgagagtatgattgaagctgagtcctctgtcaaagagaaagacatgaccaaagagttctttgagaacaaatcagaaacatggcccattggagagtcccccaaaggagtggaggaaagttccattgggaaggtctgcaggactttattagcaaagtcggtattcaacagcttgtatgcatctccacaactagaaggattttcagctgaatcaagaaaactgcttcttatcgttcaggctcttagggacaaccttgaacctgggacctttgatcttggggggctatatgaagcaattgaggagtgcctgattaatgatccctgggttttgcttaatgcttcttggttcaactcattccttacacatgcattgagttagttgtggcagtgctactatttgctatccatactgtccaaaaaagtaccttgtttctact

SEQ ID NO: 11 - DNA, 2233 bp, Polymerase A (PA) of Influenza H5N1
agcaaaagcaggtactgatccaaaatggaagactttgtgcgacaatgcttcaatccaatgattgtcgagcttgcggaaaagacaatgaaagagtatggggaagatccgaagattgaaacaaacaagttcgctgcaatatgcacacacttagaagtctgcttcatgtattcagacttccatttcattgacgaacgaggcgaatcaataattgtggaatctggtgatccgaatgcattgttgaaacaccggtttgaaataattgaaggaagagaccgagcaatggcctggacagtggtgaatagcatctgcaacaccacaggagtcgataaacccaaatttcttccggatctatacgactacaaggaaaaccgattcactgaaattggtgtgacacggagggaagttcatatatattacttagagaaagctaacaagataaaatccgaggaaacacatatccacatcttctcattcactggagaagaaatggccactaaagctgactacacccttgatgaagagagcagggcaagaatcaaaaccagactattcaccataagacaggaaatggcaagcaggggtctatgggactcctttcgtcagtccgagagaggcgaagagacaattgaagaaagatttgaaatcacagggaccatgcgtaggcttgccgaccaaagtctcccacctaacttctccagccttgaaaactttagagcctatgtggatggatttaaaccgaacggctgcattgagggcaagctttctcaaatgtcgaaagaagtgaacgccagaattgagccatttctgaagacaacaccacgtcctctcagattgcctgatggacctccctgctcccagcggtcgaaattcttgctgatggatgctctgaaattaagcattgaggacccgagccatgagggggaggggataccgctatatgatgcgatcaaatgcatgaaaacattctttggctggagagagcccaacatcatcaaaccacacgaaaaaggcataaatccaaattatctcctggcttggaagcaggtgctggcagaactccaggatattgaaaatgaggataaaatcccaaaaacaaagaacatgaagaaaacaagccaattaatgtgggcactcggagagaatatggcaccggaaaaagtggactttgaggattgcaaagacattgacgatctgaaacagtatcacagtgatgagccagagctcagatcgctagcaagctggatccagaatgagttcaacaaggcatgtgaattgaccgattcgagctggatagaacttgatgagataggggaagatgttgccccaattgagcacattgcaagtatgagaaggaactacttcacagcggaggtgtcccattgcagggctactgagtacataatgaagggggtttacataaacacagctttgctcaatgcatcttgtgcagccatggatgacttccaactgattccaatgataagcaaatgcagaacaaaagaaggaaggaggaggacaaacctgtatgggttcattgtaaaaggaaggtcccatttgagaaatgatactgacgtggtgaactttgtgagtatggaattctcccttactgacccaaggctggagccacacaaatgggaaaagtactgtgttcttgaaataggggaaatgctcttgcggactgcaataggtcaagtgtcaaggcccatgttcctgtatgtgagaactaacggaacctcaaaaattaagatgaaatgggggatggaaatgaggcgctgccttcttcaatctcttcaacagattgagagcatgatcgaggctgagtcttctatcaaagagaaagacatgaccaaagagttctttgaaaacagatcggagacatggccaattggagagtcacctaagggagtggaggaaggctccatcgggaaggtgtgcagaaccttactagcaaaatctgtgttcaacagcctatattcatctccacaactcgaaggattttcagctgaatcgagaaaactactactcattgttcaagcacttagggacaacctggaacctggaaccttcgatcttgaagggctatatggagcaattgaggagtgcctgattaatgatccctgggttttgcttaatgcatcttggttcaactccttcctcacacatgcactaagatagttgtggcaatgctactatttgctatccatactgtccaaaaaagtaccttgtttctact

SEQ ID NO: 12 - DNA, 66 bp, Artificial
  siDNA against Influenza A
ttttttgctaagttttttgctaagagttggggctggatttttcctccccatactctttcattgttt

SEQ ID NO: 13 - DNA, 54 bp, Artificial
  siDNA against Influenza A
ttttgcttttggggatgggacttgatttttcatctccattccccatttcatttt

SEQ ID NO: 14 - DNA, 54 bp, Artificial
  siDNA against Influenza A
gtgcttcttattgagctgtgtttgatttttcaggcactcctcaattgcttcata

SEQ ID NO: 15 - DNA, 54 bp, Artificial
  siDNA against Influenza A
ttggtcttcggatgtttatcttaccttttggtcggattgacatccattcaaatg